(19) United States
(12) Patent Application Publication
(10) Pub. No.: US 2011/0092890 A1
(43) Pub. Date: Apr. 21, 2011

(54) MICROCLIMATE MANAGEMENT SYSTEM

(75) Inventors: Martin W. Stryker, Kalamazoo, MI (US); Santoshkumar Balakrishnan, Portage, MI (US)

(73) Assignee: STRYKER CORPORATION, Kalamazoo, MI (US)

(21) Appl. No.: 12/899,059

(22) Filed: Oct. 6, 2010

Related U.S. Application Data
(60) Provisional application No. 61/253,485, filed on Oct. 20, 2009.

(51) Int. Cl. A61M 37/00; F25B 29/00; A41D 13/12

(52) U.S. Cl. 604/23; 165/60; 165/222; 2/69

(57) ABSTRACT

A microclimate management sheet, for placing against a person's body, includes a flexible generally fluid impermeable first layer, a flexible generally fluid impermeable second layer with a gas permeable region for facing a patient's body. The first layer is joined with the second layer in a manner to form a chamber between the first layer and the second layer. An inlet operative to be in fluid communication with a supply of gas and in fluid communication with the chamber is provided for delivering a gas to the chamber, with the gas permeable region for directing the gas the patient's body.

### CROSS-REFERENCE TO RELATED APPLICATION

This application claims priority from U.S. Pat. provisional application Ser. 61/253,485, filed Oct. 20, 2009, entitled MICROCLIMATE MANAGEMENT SYSTEM, which is incorporated by reference herein in its entirety.

### TECHNICAL FIELD AND BACKGROUND OF THE INVENTION

The present invention generally relates to a system for controlling the temperature, moisture and/or the pH a person's skin, especially a bed-ridden person's skin, such as a patient's skin.

When a person's skin is exposed to moisture, elevated temperatures, and/or acidity for prolonged periods of time, the skin can become more susceptible to injury. For example, when a person's skin, already in such a weakened state, is exposed to further stress, such as pressure, the person may be susceptible to developing a pressure sore. Pressure sores can develop when the skin is weakened and subject to pressure, for example, when a person is confined to a supine or seated position for extended periods of time. Therefore, if moisture, temperature, and/or pH of a person's skin can be controlled, for example, when the person is confined to a bed or wheelchair for prolonged periods of time, the integrity of the skin may maintained so that there is a reduced risk of developing pressure sores.

While “low-air loss” mattresses have been used to provide control over the temperature and/or moisture under a patient, when a patient is incontinent or unable to use a bed pan or leave their bed to use the toilet, the patient will often be placed in an adult diaper or be placed on an incontinence pad. However, adult diapers and incontinence pads tend to negate the effect of a low air loss mattress because both form moisture barriers between the patient's skin and the low air loss system, thus preventing air from getting to the patient's skin, which can defeat the purpose of a low air loss mattress.

Accordingly there is a need for a system that can allow air flow to the patient's skin while still providing a mechanism for absorbing body fluids.

### SUMMARY OF THE INVENTION

Accordingly, the present invention provides a microclimate management system that provides enhanced control over a person's skin temperature, moisture, and/or acidity to reduce the susceptibility of person's skin to injury.

In one embodiment, the microclimate management device comprises a sheet or flexible or conformable pad that is adapted to adjust or maintain the microclimate of a person's skin by delivering and directing temperature and/or moisture controlled air to the person's skin, which is subject to pressure, so that the device can be used as a wound prevention and treatment sheet. Further, the sheet may be adapted to deliver temperature controlled air while removing excess moisture or wetness.

For example, the sheet may also be equipped with a moisture absorbing layer to absorb bodily fluids, but which is configured to allow air flow to pass through the moisture absorbing later.

Further, the system may include a moisture detection system that can detect moisture or wetness, for example from urine, so that a person, such as a patient, won't have to spend prolonged periods of time exposed to acidic urine. For example, the moisture detection system may activate an alarm so that a caregiver can be notified when the sheet needs to be changed. This can reduce the occurrences of prolonged contact with acidic urine which can have a damaging effect, especially on delicate skin.

In one form of the invention, microclimate management sheet for placing against a person's body includes a flexible generally fluid impermeable first layer, a generally liquid impermeable, gas permeable second layer for facing a person's body, which is joined with first layer in a manner to form at least one chamber between the respective layers, and an inlet in fluid communication with the chamber for delivering a gas to the chamber for directing the gas the patient's body through the second layer.

In one aspect, the sheet comprises a mattress cover, a chair cover, an operating room table cover, or a stretcher cover. Alternately, the sheet may comprise a part of or form a garment.

In other aspects, the sheet includes a permeable layer interposed between the first and second layers, which permeable layer maintains a space between the two layers to allow the gas to flow through the sheet even when a portion of the sheet is compressed by the person's body. For example, the permeable layer may comprise a screen, an open cell foam, a three-dimensional (3D) fabric, or a drop stitch fabric.

In further aspects, the permeable layer has a substantially uniform thickness.

In yet other aspects, the sheet further includes a liquid absorbing layer over at least a portion of the second layer. For example, the liquid absorbing layer may be removably mounted to the second layer so that it can be optionally removed and then replaced with a new layer in the event that it becomes wet. Additionally, the liquid absorbing layer may be porous enough to allow gas to flow through the liquid absorbing layer or may include apertures formed therein to allow gas to flow through the liquid absorbing layer.

According to yet a further aspect, the sheet may also include a moisture detection system for detecting moisture in the liquid absorbing layer. Optionally, the sheet includes one or more sensors for sensing moisture in the liquid absorbing layer and a detector for detecting when the sensor senses moisture. For example, the sensor may be formed by conductive paths provided in the sheet, which are separated by material that is an insulator when dry but is conductive when moist or wet. Suitable materials include cotton or phase change materials. Further, the sensor may be coupled to a coil formed around a transponder, which forms a Faraday cage around the transponder when the sensor senses moisture (and therefore the transponder is not detectable by the detector) but will allow detection of the transponder when the sensors do not sense moisture. The detector may comprise a radio frequency identification device (RFID) for detecting the transponder.

In yet another form of the invention, a microclimate management system includes a generally gas impermeable first layer, a gas permeable second layer, and a generally gas impermeable third layer, with the second layer interposed between the first and the third layers and the third layer forming a skin facing surface. At least a portion of the third layer is adapted to be gas permeable so that gas can be directed through the third layer. The system also includes an inlet in fluid communication with the second layer for directing gas flow into the second layer between the first and third layers, with the second layer directing the flow of gas to the portion of the third layer that is adapted to be gas permeable for directing the flow of gas through the third layer to the person's skin, and a fluid supply device in fluid communication with the inlet for directing fluid through the inlet to the second layer.

In one aspect, the microclimate management system further includes a liquid absorbing layer over the gas permeable portion of the third layer. In a further aspect, the microclimate management system includes a plurality of sensors between the liquid absorbing layer and the gas permeable portion of the third layer for sensing when there is moisture in the liquid absorbing layer.

In yet other aspects, the first and third layers are sealed together to form two chambers, each with a gas permeable layer, and each in fluid communication with a fluid supply through the inlet. The third layer includes at least two of the gas permeable portions associated with the chambers so that gas flow into each chamber will direct fluid to a respective gas permeable portion and to a respective area of the person's skin.

For example, the inlet may include two fluid passageways, one of the fluid passageways in fluid communication with one of the chambers and the other of the fluid passageways in fluid communication with other of the chambers. Further, the inlet may be in fluid communication with two sources of gas for directing one of the gases to one of the chambers and the other of the gases to another of the chambers.

In another form of the invention, a microclimate management system includes a sheet that is adapted to control the temperature and/or moisture of a person's skin to enhance the person's comfort, for example, when the person is engaged in an activity that either raises or lowers the person's temperature and/or moisture.

Accordingly, the present invention provides a microclimate management system that provides enhanced control over a person's skin temperature, moisture, and/or acidity to either enhanced a person's comfort or reduce the likelihood of the person's skin being injured. Further, in any of the embodiments, treatments gases may be delivered using the sheet. For example, oxygen made directed to an existing wound to help heal the wound. Additionally, the whole sheet or part of the sheet may be disposable, which will help with infection control. It should be understood that part of detection system as well as the gas delivery device may be built into sheet itself, so that the sheet is a self contained unit, or can be built into an apparatus, such as a bed, stretcher, cot, operating room table, or a chair, such as a wheelchair, recliner chair, chemotherapy chairs or dialysis chairs. Alternately, where the sheet forms part of a garment, the gas delivery device may be mounted in a portable housing.

These and other objects, advantages, purposes, and features of the invention will become more apparent from the study of the following description taken in conjunction with the drawings.

### DETAILED DESCRIPTION OF THE DRAWINGS

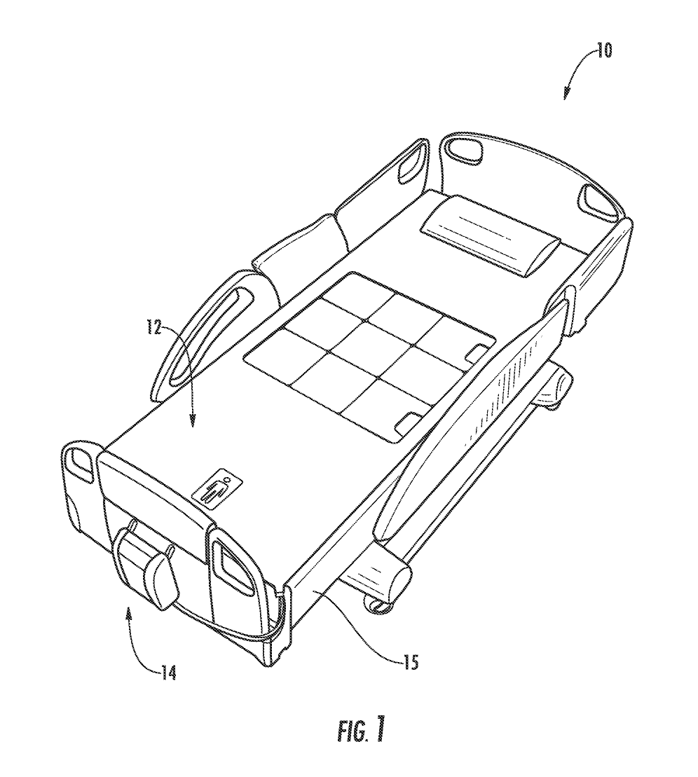

FIG. 1 is a perspective view of a patient support apparatus, namely a hospital bed with incorporating a microclimate management system of the present invention;

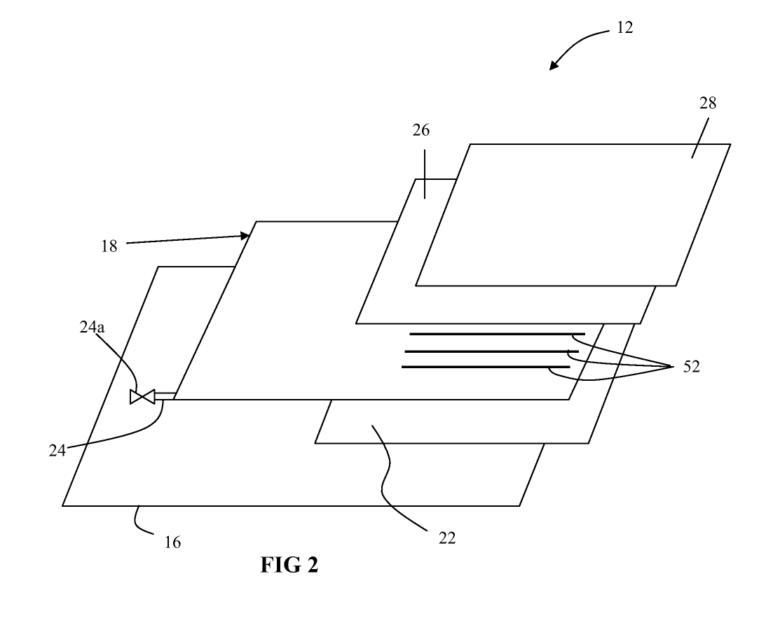

FIG. 2 is an exploded perspective view of the microclimate management device of the microclimate management system of FIG. 1;

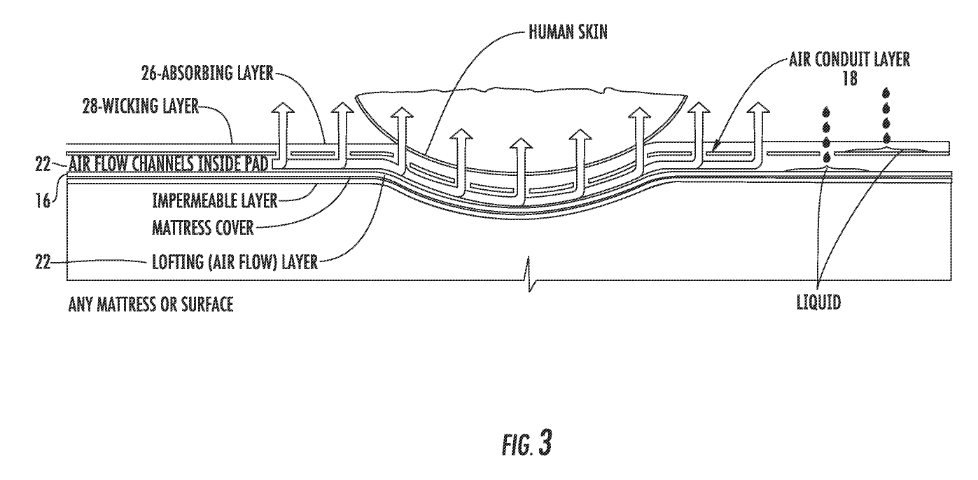

FIG. 3 is a perspective view of the air supply device of the microclimate management system of FIG. 1;

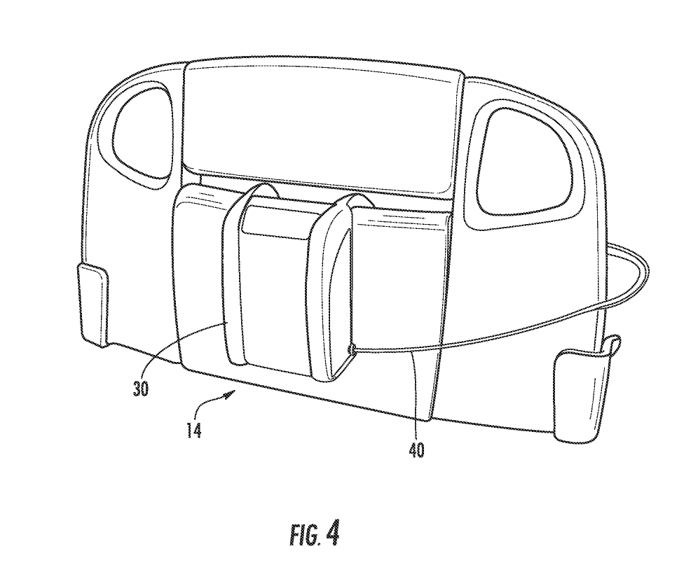

FIG. 4 is a schematic cross-section view of the microclimate management device of FIG. 2 illustrating the several layers forming one embodiment of the device and the direction of airflow through the device;

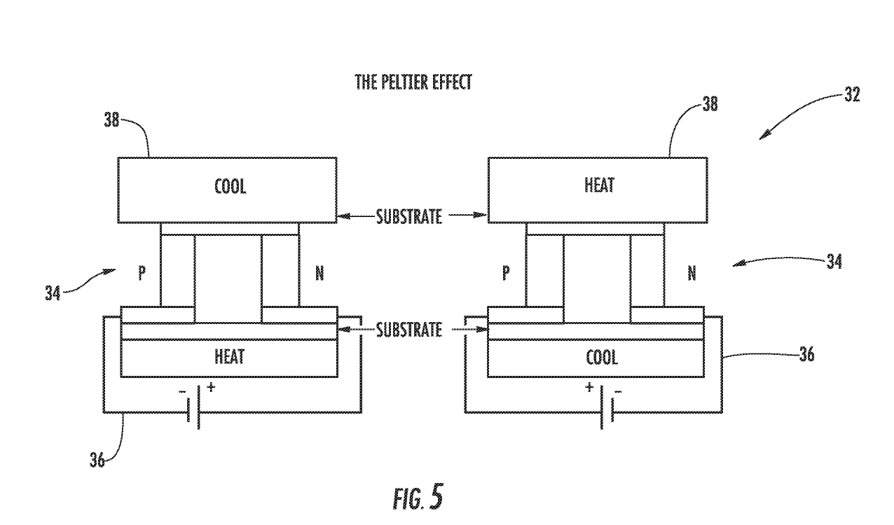

FIG. 5 is a schematic drawing of a Peltier effect device that can be used in the air supply component of the microclimate management system;

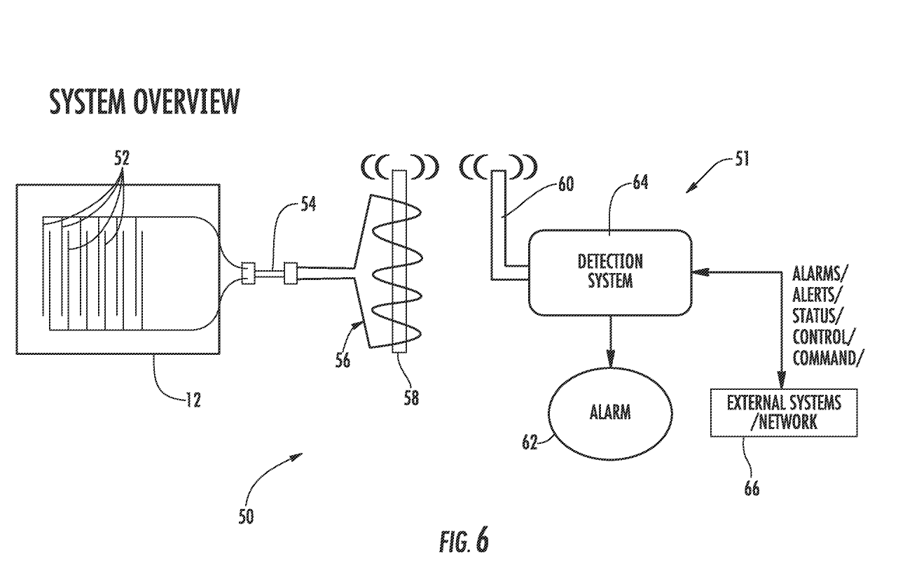

FIG. 6 is a schematic drawing of an optional moisture detection system of the microclimate management device of FIG. 2;

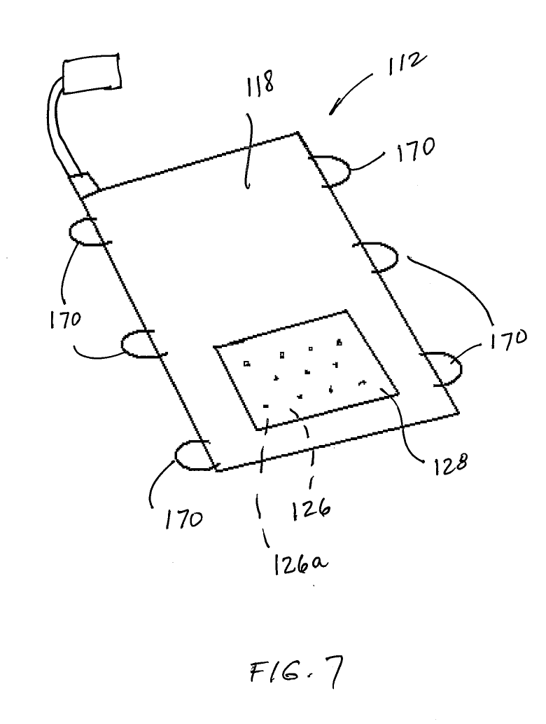

FIG. 7 is an exploded perspective view of another embodiment of a microclimate management device of the present invention in the form of a partially disposable sheet;

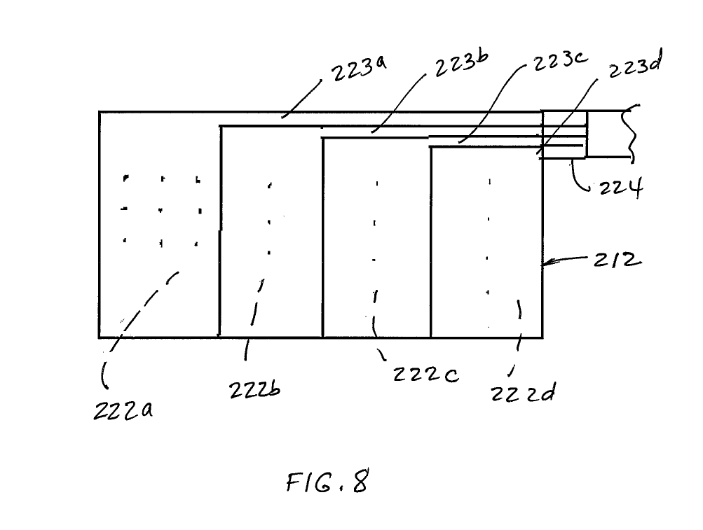

FIG. 8 is another embodiment of a microclimate management device of the present invention similar to FIG. 2 adapted to form a patient transfer device; and

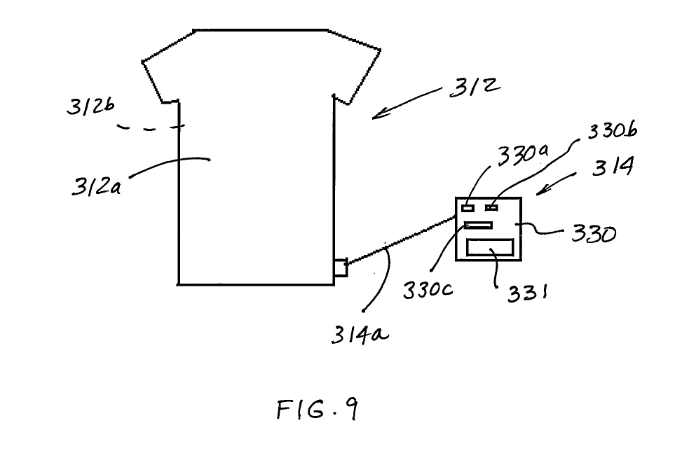

FIG. 9 is an elevation view of a garment incorporating a microclimate management device of the present invention.

### DETAILED DESCRIPTION OF THE PREFERRED EMBODIMENTS

Referring to FIG. 1, the numeral 10 generally designates a microclimate management system of the present invention. System 10 includes a microclimate management device in the form of a sheet 12, which is adapted to deliver temperature and/or moisture controlled air to a person's body, such as a patient's body, and an air delivery device 14, such as blower, which delivers air to the sheet. While much of the description that follows will make reference to a patient positioned on a bed, it should be understood that the present invention can also be used for people who are simply bedridden and not under medical care and also for people seated in chairs.

In the illustrated embodiment, sheet 12 comprise a sheet that is configured for placement on support surface, such as bed, namely a hospital bed or long term care bed, a cot, an operating room table, or a stretcher. It should be understood, as noted, that sheet 12 may also be configured for placement on a chair, such as a wheel chair, a recliner chair, a chemotherapy chair or a dialysis chair. Further, as will be described below, the sheet may be configured as a garment and worn by a person to provide a microclimate management device when the person is engaged in activity to provide comfort to the person, whose body temperature may be increased or lowered due to the activity.

Referring to FIG. 2, sheet 12 includes a base layer 16, which may be reinforced so that the sheet can be used as a transfer device, as described below in reference to FIG. 8, and an upper layer 18, which forms an air delivery layer and, further, supports optional sensors 20, which will be more fully described below. Layers 16 and 18 are formed from substantially fluid (gas and liquid) impermeable material so that when they are joined together, such as by heat sealing or the like, they form one or more chambers, more fully described below. Further, they may be made from launderable and/or disposable materials to help with infection control. For example, upper layer 18 and base layer 16 may be joined and sealed at their respective perimeters. Layers 16 and 18 may also be formed semi-permeable materials, with layer 18 having a highly permeable portion, described below. For example, suitable materials include coated nylon or coated polyester, TYVEK or GORTEX.

Additionally, at least upper layer 18 may be formed from a material that reduces the sheer stress on a patient's skin. For example, upper layer 18 may be formed from a flexible and somewhat stretchy and generally impermeable material, such as TYVEK. TYVEK tends to be a compliant and stretchy material so that when a patient moves on the sheet, the upper layer will not pull or tug on the patient's skin and instead move with the patient's skin, at least over a limited range of motion.

Positioned between base layer 16 and upper layer 18 is an intermediate layer 22, which is fluid permeable and, further, forms a space between the upper layer and the base layer to allow gas to flow through sheet 12. Sheet 12 further includes one or more inlets 24, which are in fluid communication with the chamber formed between the upper and base layers, which is used to direct gas flow into sheet 12 and, further, into permeable layer 22. Inlet 24 may be formed from a tubular member inserted and secured between upper layer 18 and base layer 16 or may be formed from the fabric forming the upper layer and/or the base layer. Furthermore, inlet 24 may include a valve 24a such as a check valve, so that if the blower is turned off, the air that is directed into the sheet 12 will remain in sheet 12 until the air is discharged through the air delivery portion of upper layer 18.

Permeable layer 22 may extend the full length of the sheet and fill the entire space between the upper and base layers or may be located for example, as illustrated in FIG. 2, only over a discrete region where a patient may be positioned so that layer 22 will maintain a space between the upper layer and the base layer at least under the patient to allow gas to flow through the sheet and under the patient. Intermediate layer 22 may be formed from a variety of different materials including a conventional lofting material, a 3-D fabric, or a screen material with off-set regions or points to allow airflow across and through the screen. For example, the screen may be molded from a polymeric material. In these forms, the intermediate layer is non-crushable so that air may flow across the intermediate layer even when a patient is laying on top of the sheet. Another suitable material for intermediate layer includes a drop-stitch, which when inflated forms a permeable layer but when un-inflated compresses back to a relatively impermeable layer. In addition, intermediate layer 22 may be formed with channels so that air is directed through sheet 12 in channels to better direct the flow of air through sheet 12 and better target specific areas of the patient's body. This may be particularly suitable if the gas that is directed into sheet 12 is a treatment gas, such as oxygen.

Additionally, upper layer 18 and base layer may be joined to the intermediate layer so that the intermediate layer prevents layers 18 and 16 from billowing or tacoing, as is know in the art. Alternatively, layer 18 may be joined at discrete locations to base layer 16, for example, by heat sealing to control the billowing and/or tacoing effect when gas is introduced in to the space between upper and base layers 18, 16.

As noted above, upper layer 18 is adapted to form an air delivery layer. For example, upper layer 18 as noted may be formed from an impermeable material or a semi-permeable material with a highly permeable portion or section. For example, highly permeable portion may be provided by apertures formed in the layer or may be formed by a section of another fabric with a larger weave so that the fabric is porous. Optionally, where layer 18 is formed from a substantially impermeable layer, layer 18 may be provided with a semi-permeable portion, such as GORTEX insert, which allows air to pass through but prevents liquid from flowing back through the layer, for example, in the case of an incontinence episode. The permeable portion or region may be confined to a single or multiple discrete locations. For example, it may be desirable to have one location that generally would coincide with the hip and buttock region of a patient and another at the heel area of the patient, where the pressure on a patient's skin may be highest depending on the patient's body type and the mattress configuration.

Optionally positioned on top of the permeable portion of upper layer 18 adjacent the hip and buttock area of the patient may be an absorbent layer 26, which is used to collect bodily fluids, such as urine, from the patient, and in a wicking layer 28, which is positioned over the absorbent layer to minimize of not eliminate contact between the patient's skin and the moisture contained in the absorbent layer 26. For example, a suitable wicking layer includes the wicking layer commonly used in diapers. Absorbent layer 26 and wicking layer 28 are integrated with upper sheet 18 for example by stitching, by welding, by an adhesive, or the like. In this manner, when airflow is introduced into inlet, the air will flow through the space between the upper and lower sheets and into the intermediate layer 22, which will disperse the flow of air and direct the flow of air through the permeable portion of the upper layer. To facilitate the flow of air through the absorbent layer, absorbent layer 26 optionally includes a plurality of apertures to assure that airflow is delivered to the desired location of the patient's body. The apertures may be arranged in a uniform pattern or a non-uniform pattern.

Referring to FIG. 3, when a patient is positioned on sheet 12, and air is directed into the sheet, air will flow through the space between the upper and lower layers 16 and 18 and into permeable layer 22, which will maintain the space between the upper and lower layers so that air can flow through the sheet and underneath the patient's body. The air will then flow through upper layer 18 through the openings provided in or formed in layer 18 so that air will be directed into the absorbing layer which in turn will direct air to the patient's skin through the wicking layer. As will be described below, the temperature of the airflow is controlled to maintain the temperature of the patient's skin in a desired temperature range and, further, will enhance the wicking away of moisture from the patient's skin to maintain the patient's skin to a desired moisture level or dryness. Should a patient's body release fluids, for example, if the patient urinates, the fluid will pass through the wicking layer 28 and into the absorbing layer 26, which liquid will generally remain trapped between the wicking layer and the upper layer 18 so that the fluid will not make contact with a mattress positioned below the base layer 16. Where upper layer 18 has openings instead of a liquid impermeable, gas permeable layer, even should the liquid pass through the openings provided in upper layer 18 into the permeable layer 22, the base layer 16 will prevent the liquid from making contact with the mattress. At this point the sheet may be disposed of and replaced with a new sheet. Where upper layer includes a liquid impermeable portion, the upper surface of the sheet may be cleaned and only the absorbent layer and wicking layer need be disposed of, as will be more fully described in reference to FIG. 8.

To deliver air flow into sheet 12, as noted above, system 10 includes an air delivery device, such as a blower. The blower may be used to deliver air to sheet 12 and/or to deliver a treatment gas, such as oxygen, for example in the case where a patient has already developed a pressure sore. Therefore, the blower may be in fluid communication with a supply of gas treatment. In addition, the air delivery device may include more than one blower, for example, with one blower for delivering air and the other for delivering a treatment gas. Further, the blower may be housed in a separate housing (30) as described below, or made be built on to a support surface, such as bed, for example, in the head or footboard.

In the illustrated embodiment, the blower is located in housing 30, which is adapted to removably mount to a support surface, such as a hospital bed, including to the footboard, and which supports a controller for operating the blower. The blower may be a conventional blower and further optionally blows air across a heating/cooling unit 32 (FIG. 5) so as to provide cool or warm air. In the illustrated embodiment, heating/cooling unit 32 is a conventional heating/cooling unit with a Peltier effect device 34, which will either heat or cool air depending on the polarity of current provided by the controller. The application of a direct current supply to heating/cooling unit 32 causes one of the first and second sides 36, 38 of the device to heat up and the other of the first and second sides to cool. Reversing polarity of the direct current causes a corresponding reversal in which of the two sides 36, 38 is heated or cooled. This reversing of polarity may be provided by a switch or transistor which is controlled by the controller based on input from one or more user actuatable devices, such as buttons or touch screens, provided at housing 30 or at some other location, such as a pendent or remote control device, which is in communication with the controller in housing 30.

As best seen in FIG. 4, air delivery device 14 is in fluid communication with sheet 12 via a conduit 40 formed from conventional plastic tubing, which may comprise a single lumen tubing or multiple lumen tubing, for example, should the sheet be segregated into multiple air delivery regions, more fully described below. Further, as noted, housing 30 may support user interface devices, such as buttons or touch screen or the like, to allow a user to control the operation of the blower and further to select the temperature and, optionally, even further the flow rate of the air flowing into the sheet. In addition, housing 30 may house a power source, such a battery, including a rechargeable battery, for powering the blower and the heating and cooling device. Alternately, or in addition, the controller, blower and heating and cooling unit may be powered by the bed electrical system or from a wall outlet.

As noted above, sheet 12 may incorporate a moisture detection system 50, which includes a detector and a sensing system. Moisture detection system 50 may be configured as an inductive detection system, and at least a portion of the detection system may be located in housing 30 or may be located for example in the footboard of the bed. In the illustrated embodiment, the sensing system includes a plurality of sensors, which are formed by a conductive path 52, for example, conductive wires or screen printed conductive layers, which are located in sheet 12. Alternately, conductive paths may be formed by two or more stacked conductive screens. Referring to FIG. 2, the conductive paths may be located below moisture absorbing layer 26 and further may be, for example, printed on upper layer 18.

As best seen in FIG. 5, in the illustrated embodiment, conductive paths 52 are arranged into what is commonly known as a comb capacitor arrangement, which are then connected via connecting cable 54 to a coil 56, which is wound around a transponder 58. Cable 54 may comprise a two or three wire cable with two of the wires connecting to the terminals of the conductive paths provided on the sheet and with the third wire used as a ground connection. The conductive paths may be arranged in a common plane or may be stacked but are separated by a material that is non-conductive when dry but conductive when wet. For example, a suitable material is a cotton material or a gel, which when exposed to moisture or wetness will dissolve. Also, something as simple as a powder, such as cornstarch, may be used to separate the conductive paths but which when wet will become conductive. In this manner, when moisture absorbing layer 26 is moist or wet, the moisture will cause closure of contacts between the conductive paths, which shorted paths create a loop via the connecting cable and coil of wire around the transponder to thereby create a Faraday cage. When the contacts are closed, the transponder will not operate from within the Faraday cage. As will be more fully described below, when the detection system cannot read or see the transponder, this indicates that the moisture absorbing layer or pad is wet or moist and suggests an incontinence episode or fluid discharge onto the sheet.

A suitable transponder is a passive RFID-type device. These devices can operate at low frequency, very high frequency, or ultra-high frequency ranges. For example, the transponder may be in a form of a card, small stick, key fob, disc, or flexible printed electronics or the like, with embedded antenna and associated circuitry. Suitable RFID-type devices are available from Texas Instruments.

The detector comprises an antenna 60, which may be housed for example in the footboard or headboard of the bed or in housing 30 described above. Optionally, the electromagnetic energy transmitted from the antenna may be used to “wake-up” or activate the transponder. The detection system may also generate a signal to initiate an alarm or an alert 62, such as an alarm or alert device on the bed or through a bed network system to a remote alarm when moisture is sensed by the sensors. For example, the hospital bed may include a nurse call system or a communication system that allows the alarm to be forwarded onto a location remote from the bed, for example to a nurse call station so that the hospital staff may be notified that the sheet is wet so that appropriate action may be taken. Additionally, these incontinence episodes may be recorded at the bed, for example in a bed based-computer, or at a remote station, which can be used to track the care of a patient and/or to track whether the patient is properly hydrated.

A suitable alarm may consist of a visual indication, an audible indication, or a combination of visual and audible indication. For example, visual indicators may include LED and LCD displays with specific messages or pattern or patterns of flashing lights, etc. Audible indications may include monotonic or polyphonic sounds, recorded voice messages, repetitive sequences of a pattern of sounds, etc. including a repetitive sequence of the patterns of sounds.

In the illustrated embodiment, the detector is essentially an RFID reader type device 64 capable of reading RFID transponders at respective frequency ranges. Further, the reader may be capable of reading information from the tag (if the tag is provided with additional information for transmission) as well as writing information to the tag, as is commonly known in RFID-type systems. Further, as noted, it may be capable of providing, for example via suitable electronic interface, information about the tag to an external device system or network and, further, may activate a secondary circuit for alarming in the invent of an incontinent episode.

For example, a suitable external system device or network 66 may include a computer or a network device or other electronic boards and circuits including the bed network noted above. In this manner, detection system 50 may provide messages, alerts, commands, and other controls within other system devices in order to manage the incontinence episode detected by the system.

As described, system 10 may incorporate an air delivery device 14 for delivering warm and/or cold air to sheet 12. Alternately, air delivery device may simply comprise a conduit for delivering air to the sheet from an onboard air supply, such as described in copending application Ser. No. 12/057,941, filed Mar. 28, 2008, entitled PATIENT SUPPORT WITH UNIVERSAL ENERGY SUPPLY SYSTEM, which is incorporated by reference in its entirety, which is commonly owned by Stryker Corporation of Kalamazoo, Mich.

As an alternate or in addition to moisture detection system 50, system 10 may incorporate a visual based moisture detection system. For example, sheet 12 may incorporate a layer or strips of litmus paper, which when moistened with urine will change color due to the acidity in urine. A fiber optic sensor may also be located in sheet, which is coupled to a controller, for example, in housing 30 or the controller on the bed, which can initiate an alarm or alert if the change in color is detected. Further, the degree of color may indicate whether the patient is sufficiently hydrated or not. The litmus paper may also be used to detect other conditions, for example, it may be configured to detect the presence of certain proteins in the urine, which will indicate a patient condition. For example, it may be configured to detect the presence of proteins that indicate pregnancy. Therefore, sheet 12 may also be configured as a diagnostic tool as well or instead of a wound treatment prevention device.

Referring to FIG. 7, sheet 112, which is of similar construction to sheet 12 may include a removable and replaceable wicking layer 128 and moisture absorbing layer 126 so that the wicking layer and moisture absorbing layer may be removed and replaced so that the sheet may be continue to be used by the same patient even after an incontinence occurrence, as noted above. In order to prevent contamination of the upper sheet 118, wicking layer 128 and moisture absorbing layer 126 may incorporate a bottom layer of moisture impermeable, gas permeable material, such as GORTEX. so that even if the moisture absorbing layer 126 is saturated with a fluid such as urine, airflow can still be maintained through the various layers to reach the patient but prevent contamination of the upper layer of sheet 118. In this manner, only a portion of the sheet 112 need be disposed of, so that the sheet 112 is partially disposable and the remaining portions of the sheet remaining reusable without necessarily requiring cleansing, cleaning, or disinfecting. In this embodiment, it may be preferable to form the upper wicking layer, moisture absorbing layer 126 and liquid impermeable but gas permeable lower layer 126a as a self contained pad that can be applied to sheet 112 over the at least gas permeable region of upper layer 118.

In additional, sheet 112 (as well as sheet 12) may incorporate hand holes 170 along its peripheral edges to allow sheet to be used as a patient transfer device after the sheet 112 has been disconnected from its air supply device. For example, handles 170 may be formed from straps, which are welded or sewn or otherwise attached to the perimeter of the sheet. While the straps are shown at the sides of the sheet, it should be understood that they may be located at the foot or head end of the sheet.

Optionally, as best seen in FIG. 8, sheet 212 may include an intermediate layer 222 that is segregated, for example by seams formed, for example by welding, between the upper layer 218 and the base layer 216 with each region 222a, 222b, 222c, and 222d of intermediate layer 222 providing a fluid directing function—either independent from the others or together with one or more areas so that different parts of the body may be treated with air of differing temperatures. Each region 222a, 222b, 222c, and 222d may therefore be in fluid communication with a respective fluid passageway 223a, 223b, 223c, and 223d for coupling via inlet 224 to either discrete conduits or a conduit with multiple lumens, with each lumen being coupled to an air supply. For example, each lumen may be in fluid communication with an air supply device, which is controlled by a controller. Alternately, a single air supply device may be used and control through the respective lumens may be achieve by way of a plurality of valves, such as solenoid valves, or a valve manifold, for example a solenoid valve manifold, with the control opening and closing the valves based on input from a user or treatment protocol, for example, stored in the controller. Where two or more air delivery devices are used, the air delivery devices may be separately controlled and further may each have a heating/cooling unit so that air may be delivered to one area at one temperature, and air at a second temperature may be supplied to another area. For example, one air delivery device may provide cold air with the other delivery device providing hot or warm air. In this manner, the sheet may be customized to supply different temperatures along its length and over different regions of the sheet.

In a similar manner, each region 222a, 222b, 222c, and 222d of sheet 212 may have a different pattern of holes or apertures or different permeability, again to tailor the airflow to the patient's body. In addition, to allow a user to modify the sheet to a particular patient's needs, the sheet may have removable covers (e.g. removable impermeable covers) that close off the respective areas as air delivery areas so the user may select which area to open. Furthermore, one or more regions may include a moisture detection system of the type disclosed above. Again, sheet 212 may be fully disposable or may have disposable portions, such as described in reference to sheet 112.

Any of the above sheets may also incorporate a fecal incontinence detection system. Fecal matter if left in contact with a patient's skin has even more potential to cause injury than urine. In one form, a sensor may be incorporated into the sheet, for example under or adjacent the hip and buttock region of the patient, which forms a sniffer that can detect the presence of a gas, for example methane. The sensor is coupled to a controller, such as the controller in housing 30, or a controller on the bed. When the controller detects that a gas associated with a fecal incontinence episode is sensed by sensor, the controller will activate an alarm or alert in a similar manner as described in reference to the moisture detection system. Alternatively, the sensor may be external to the sheet, with the sheet have a blower to blow air through the sheet as exhaust, which exhaust is directed to the sensor either directly or through a conduit, such as a flexible tube. It should be understood that the system may also be used to detect other gases, which are indicative of other patient conditions.

While each of the embodiments of the sheet have been described in reference to providing a moisture absorbing layer and a wicking layer as well as an optional moisture detection system and an optional fecal incontinence detection system, it should be understood that the respective sheets may be configured simply as an air delivery device for delivering air to the patient without the wicking layer and/or moisture absorbing layer, and also without the detection systems. Furthermore, any of the sheets may include extended portions for wrapping over a patient, for example, which may be particularly suitable for male patients. Additionally, the sheets may be formed as a warming or cooling blanket to be used in a bed, a stretcher, a cot, an operating table, or a chair and further have non-medical applications, such as for use on a golf cart.

It can be appreciated from the above description, the disposable nature of the sheets of the present invention may help with infection control. The air delivery function can be used in wide variety of applications beyond wound prevent or treatment and can be used can be used on top of the patient to control body temperature, for example, to treat hypothermia or heat stroke to help the patient in post operative recovery. Optionally, the blower may also be used to inflate a low air loss mattress and deliver temperature controlled air through the sheets.

Referring to FIG. 9, the sheet of the present invention may also be configured or incorporated into a garment 312. For example, the sheet may be incorporated both into a front panel of the garment 312a as well as a rear panel of the garment 312b so that airflow can be directed both to the patient's chest and stomach area and/or back area. While the garment 312 is illustrated as a top, such as a vest or tunic, the garment may be configured as a pair of pants, shorts, and or a long sleeve top, or may be configured as a body suit, for example. Garment 312 may be worn by a wide variety of people, including surgeons, nurses, pilots, construction workers, hunters, hazmat workers, all of whom may need to be cooled or warmed depending on the environment.

Each panel 312a and 312b may be formed in a similar manner to sheets 12 or 212 each with either a single region through which airflow is directed or multiple regions through which airflow is directed to optionally provide airflow at different temperatures to the different regions of the body. It should be understood that the outer facing side of each panel is formed from the generally fluid impermeable fabric or material, while the inwardly facing side of the respective panel may be formed from a generally fluid impermeable material or fabric with one or more permeable regions to provide airflow through the sides toward the person wearing the garment. Alternately, the inwardly facing sides of each panel may be formed from a gas permeable material so that airflow is directed across the entire surface of the patient's chest and back area. Further, as in the case of the sheets described above, the gas permeable portions of the respective panels may have different density of openings or apertures to allow greater airflow to certain regions of the body. Further, each of the respectively inwardly facing sides of the panels may include a layer of wicking material, such as wicking layers used in diapers, which allows air to flow through to the person's skin but draws moisture away from the person's skin.

In a similar manner to the previous embodiments, panels 312a and 312b are connected to a fluid supply delivery device 314 such as a small blower or fan, which is powered by a battery, such as a rechargeable battery. It should be understood that the blower may also be powered by an external power supply, such as a wall socket or, in the case of a surgeon, by the electrical system of the operating table.

The blower, for example, may be in fluid communication with the respective panels via a conduit 314a, such as a flexible tube with one or more lumens, and/or may be incorporated into the garment so that the blower directs air directly into the chamber or chambers. Optionally, the blower may have a tube that can be selectively inserted into one of the chambers, for example through a self sealing opening, so that the wearer can select where they wish air to be directed. Further, like the sheets, the blower may be configured to deliver cool or warm air or both. For an example of a suitable heating/cooling device reference is made to the first embodiment.

To reduce power consumption, the blower may use an ice pack or other phase change materials, such as a gel, in lieu of the Peltier effect device described above, which can hold a temperature for a prolonged period of time when activated, and in some case up to 8 hours. The blower then directs air across the phase change material to either heat or cool the air being delivered to the chamber or chambers of garment 312.

Optionally, as noted air delivery device 314 may be integrated into the garment either in a sack or pocket formed in one of the panels or may be simply housed in a housing 330 that can be suspended from the patient's body using one or more straps, belts, or the like. Similarly, the housing may further support user actuatable interface devices, such as one or more buttons 330a and 330b, which either directly turn on or off the respective blower or control the blower via a controller, such as a microprocessor. In addition, as noted, housing 330 may support an energy supply, such as a battery 331 for powering the blower, which may be rechargeable. Additionally, therefore, housing 330 may support a display or other indicator 330c, which indicates the status of the battery.

Additionally, the housing containing the fluid delivery devices of any of the above embodiments may incorporate ports, such as USB ports, for plugging other devices into the housing for providing additional functionality, for example, for powering other devices, for example including lights, worn for example by a surgeon.

While several forms of the invention have been shown and described, other changes and modifications will be appreciated by those skilled in the relevant art. Therefore, it will be understood that the embodiments shown in the drawings and described above are merely for illustrative purposes, and are not intended to limit the scope of the invention which is defined by the claims which follow as interpreted under the principles of patent law including the doctrine of equivalents.

The embodiments of the invention in which an exclusive property right or privilege is claimed are defined as follows:

1. A microclimate management sheet for placing against a person's body, said sheet comprising:
a flexible generally fluid impermeable first layer;
a flexible generally fluid impermeable second layer with a gas permeable region, and said first layer joined with said second layer in a manner to form a chamber between said first layer and said second layer, and said second layer for facing a patient's body; and
an inlet operative to be in fluid communication with a supply of gas and in fluid communication with said chamber for delivering a gas to said chamber, and said gas permeable region for directing the gas the patient's body.

2. The microclimate management sheet according to claim 1, wherein said sheet comprises a mattress cover, a chair cover, an operating room table cover, or a stretcher cover.

3. The microclimate management sheet according to claim 1, wherein said sheet is incorporated into or forms a garment.

4. The microclimate management sheet according to claim 1, further comprising a a permeable layer, said permeable layer interposed between said first and second layers.

5. The microclimate management sheet according to claim 4, said permeable layer comprising a screen, an open cell foam, a three-dimensional (3D) fabric, or a drop stitch fabric.

6. The microclimate management sheet according to claim 4, wherein the permeable layer has a substantially uniform thickness.

7. The microclimate management sheet according to claim 1, further comprising a liquid absorbing layer over at least a portion of said second layer.

8. The microclimate management sheet according to claim 7, wherein said liquid absorbing layer is removably mounted to said second layer.

9. The microclimate management sheet according to claim 7, further comprising a moisture detection system for detecting moisture in said liquid absorbing layer.

10. The microclimate management system according to claim 9, wherein said moisture detection system includes a transponder.

11. The microclimate management system according to claim 10, further comprising a Faraday case, said transponder being located in said Faraday cage.

12. A microclimate management system comprising:
a generally gas impermeable first layer;
a gas permeable second layer;
a generally gas impermeable third layer, said second layer interposed between said first and said third layers, said third layer forming a skin facing surface, and at least a portion of said third layer having gas flow openings at said skin facing surface wherein at least a portion of said third layer is adapted to be a gas permeable layer;
an inlet in fluid communication with said second layer for directing gas flow into said second layer, and said second layer directing the flow of gas to said openings for directing the flow of gas through said third layer at said facing surface; and
a fluid supply device in fluid communication with said inlet for directing fluid through said inlet to said second layer.

13. The microclimate management system according to claim 12, further comprising a liquid absorbing layer over said gas permeable portion of said third layer.

14. The microclimate management system according to claim 13, further comprising a plurality of sensors between said liquid absorbing layer and said third layer, and a detector, said detector in communication with said sensors to detect when said sensors sense moisture in said liquid absorbing layer.

15. The microclimate management system according to claim 14, wherein said detector comprises an RFID reader, and said sensors form at least one RFID tag.

16. The microclimate management system according to claim 12, wherein said second layer forms at least two discrete chambers, and said third layer includes at least two of said gas permeable portions, said chambers associated with said gas permeable portions wherein each chamber directs fluid to a respective gas permeable portion.

17. The microclimate management system according to claim 16, wherein said inlet includes two fluid passageways, one of said fluid passageways in fluid communication with one of said chambers and the other of said fluid passageways in fluid communication with other of said chambers.

18. The microclimate management system according to claim 17, wherein said inlet is in fluid communication with two sources of gas for directing one of said gases to one of said chambers and the other of said gases to another of said chambers.

19. A method of treating a patient's skin comprising:
controlling the temperature of a gas;
absorbing moisture from the patient's body into a layer of moisture absorbent material; and
directing the temperature controlled gas to a person's skin through the absorbent material.

20. The method according to claim 19, further comprising detecting the presence of moisture in the layer of moisture absorbent material.

21. The method according to claim 20, wherein said detecting the presence of moisture includes sensing the presence of a transponder.
